(19) United States
(12) Patent Application Publication
(10) Pub. No.: US 2016/0129229 A1
(43) Pub. Date: May 12, 2016

(54) Moisturizing Finger Cover

(71) Applicant: Marshall J Pettygrove, Hailey, ID (US)

(72) Inventor: Marshall J Pettygrove, Hailey, ID (US)

(21) Appl. No.: 14/538,543

(22) Filed: Nov. 11, 2014

(51) Int. Cl. A61M 35/00; A61F 13/10

(52) U.S. Cl. CPC A61M 35/00; A61F 13/104

(57) ABSTRACT

An apparatus worn on the finger that can prevent cracked skin. In order to do so, the apparatus consists of a finger cover, an attachment mechanism, and a quantity of lotion. The finger is inserted into the finger cover through an open end. Next, the finger cover is secured with an attachment mechanism. When secured, the finger will be in contact with the quantity of lotion placed at a closed end of the finger cover. The finger cover is made of transparent plastic. As a result of being transparent, the finger cover has low visibility when worn. The apparatus can be worn when engaging in daily activities since the plastic is lightweight and durable. Since the finger cover is a loose fitting finger cover, the user can wear the apparatus comfortably.

### FIELD OF THE INVENTION

The present invention relates generally to field of medical and health care apparatus. More specifically, the present invention is a device that can be used to heal or prevent a finger of a user from cracking.

### BACKGROUND OF THE INVENTION

As witnessed by many individuals, cracked skin can be painful and can also disrupt the daily activities of an individual. Cracked skin can occur due to non-medical reasons and also due to medical disorders of a particular individual. Nerve damage, reduced sweating, and dehydration are some of the mentioned medical disorders that can result in cracked skin. Abrupt changes in temperatures, cold weather, and excessive exposure to hot water, detergents, or other harsh chemicals can be listed as some of the non-medical causes of cracked skin. In both instances, cracked skin can be equally uncomfortable for an individual.

On most occasions, cracked skin is ignored as to being a temporary issue. However, seeking treatment is essential, especially since cracked skin can be symptoms of life threatening conditions. As an example, cracked skin can be symptoms of diabetes, hormonal imbalance, hypothyroidism, malnutrition, and nerve damage. Additionally, other unfavorable skin symptoms can also occur along with cracked skin. As an example, blisters, itchy skin, redness, and scaly skin can occur along with cracked skin.

Even though complicated remedies are utilized to heal cracked skin on the body, less attention is given to cracked skin on fingers. This can be disadvantageous since fingers are consistently used throughout a person's daily activities. Applying moisturizer has been the most favored and recommended method of preventing cracked skin. Even though applying moisturizer is simple and effective, it has certain drawbacks too. As an example, a layer of applied moisturizer can dry up or rub off within a short time period. If the atmospheric air is extremely dry, the layer of moisturizer can dry within a short time period. Furthermore, in order to maintain moisture, a user needs to apply moisturizer at regular intervals. Such procedures may not be possible with the busy schedules in today's society. For instance, regularly applying moisturizer may not be possible for individuals who frequently travel. Furthermore, if an individual works outdoors for the majority of the time, any applied moisturizer will dry or rub off within a short time period. Therefore, maintaining moisturizer throughout the day can be challenging.

The objective of the present invention is to address the aforementioned issues. In particular, the present invention is an apparatus that can be worn on a finger to heal cracked skin or prevent the skin on the finger from cracking. The effectiveness of the present invention allows the user to wear the present invention for long periods of time with convenience. For instance, the present invention can even be worn overnight with great effectiveness. As a result, the need to apply moisturizer at regular intervals is eliminated. Another advantage of the present invention is its mobility. In comparison to traditional moisturizer carrying methods, the present invention is advantageous in terms of mobility. More specifically, the design of the present invention allows the user to carry the present invention conveniently. The ease of carrying the present invention can be especially beneficial in instances with limits for carrying moisturizers, such as when boarding an airplane. By utilizing the present invention, the user can heal cracked fingers or keep their fingers from cracking due to non-medical reasons and also medical disorders.

### BRIEF DESCRIPTION OF THE DRAWINGS

FIG. 1 illustrates a perspective view of the present invention.

FIG. 2 illustrates a top view of the present invention.

FIG. 3 illustrates the open end of the present invention.

FIG. 4 illustrates another side view of the present invention, wherein both the attachment mechanism and the open end are illustrated.

FIG. 5 illustrates a finger being partially inserted through the open end of the present invention.

FIG. 6 illustrates the finger being completely inserted into the finger cover of the present invention.

### DETAIL DESCRIPTIONS OF THE INVENTION

All illustrations of the drawings are for the purpose of describing selected versions of the present invention and are not intended to limit the scope of the present invention.

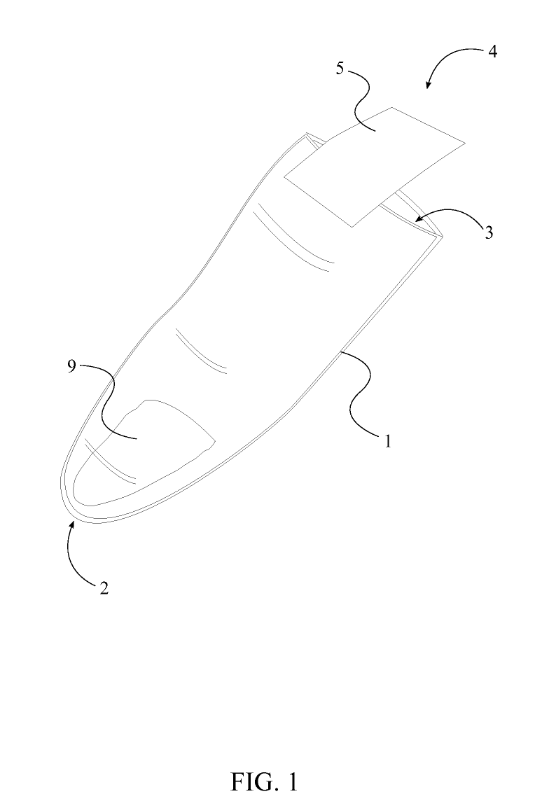

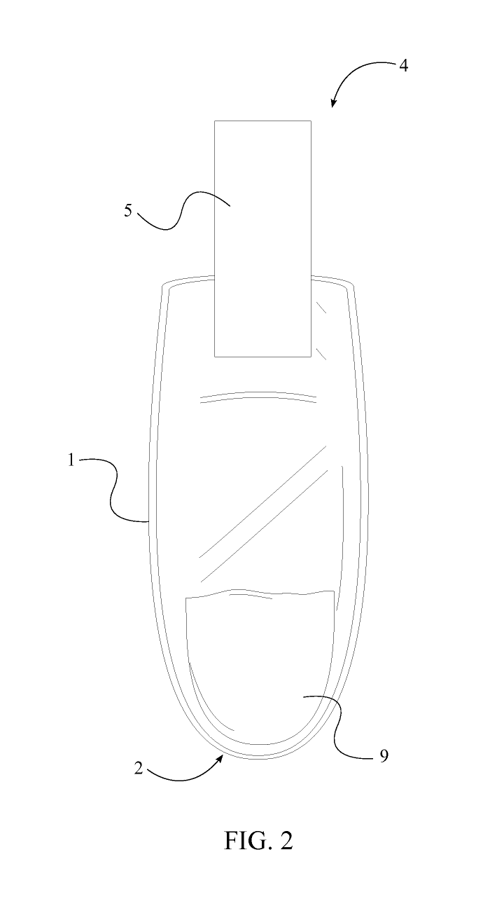

The present invention is an apparatus that can be used to maintain moisture on a finger. As a result of maintaining the moisture, unfavorable circumstances such as cracked skin or damaged skin can be healed or prevented. In order to do so, the present invention comprises a finger cover 1, an attachment mechanism 4, and a quantity of lotion 9 as illustrated in FIG. 1 and FIG. 2. A finger which requires moisturizing is inserted into the finger cover 1. The attachment mechanism 4 is utilized in order to secure the present invention with the finger of the user. The quantity of lotion 9 is then utilized to provide moisture to the finger.

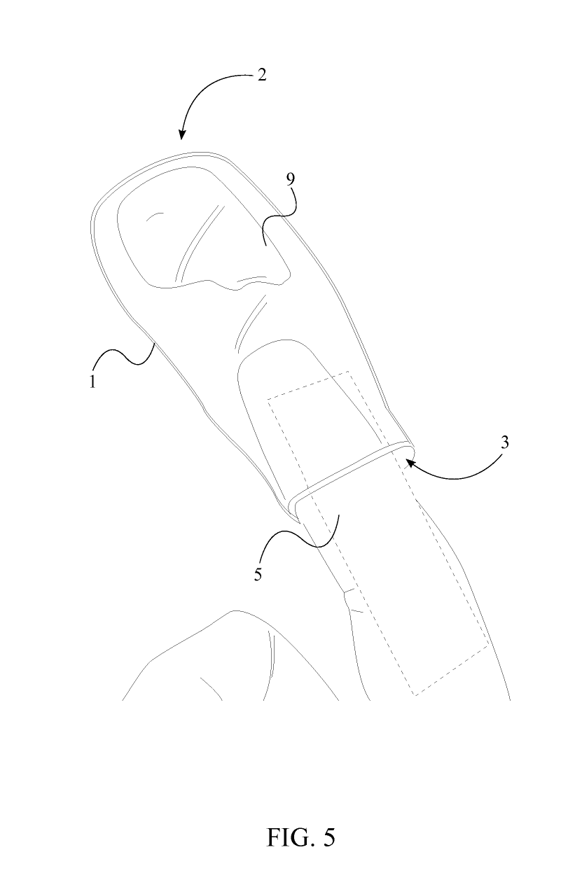

As illustrated in FIGS. 1-5, the finger cover 1 comprises a closed end 2 and an open end 3. The shape of the finger cover 1 allows the user to insert the finger and keep the finger inside the finger cover 1, comfortably. The finger cover 1 is designed as a loose fitting finger cover such that the finger has a full range of motion when the present invention is worn. Additionally, the loose fitting finger cover allows air to circulate within the finger cover 1. The finger is inserted through the open end 3. The closed end 2 and the open end 3 are positioned opposite to each other along the finger cover 1. As a result, when the present invention is worn, the closed end 2 of the finger cover 1 is positioned adjacent to the top of the finger the present invention is worn on. The quantity of lotion 9 is initially placed at the closed end 2 in the preferred embodiment of the present invention. More specifically, the quantity of lotion 9 is positioned within the finger cover 1 adjacent to the closed end 2. However, in another embodiment of the present invention the quantity of lotion 9 can be positioned elsewhere within the finger cover 1. For instance, the quantity of lotion 9 can be spread across the interior of the finger cover 1.

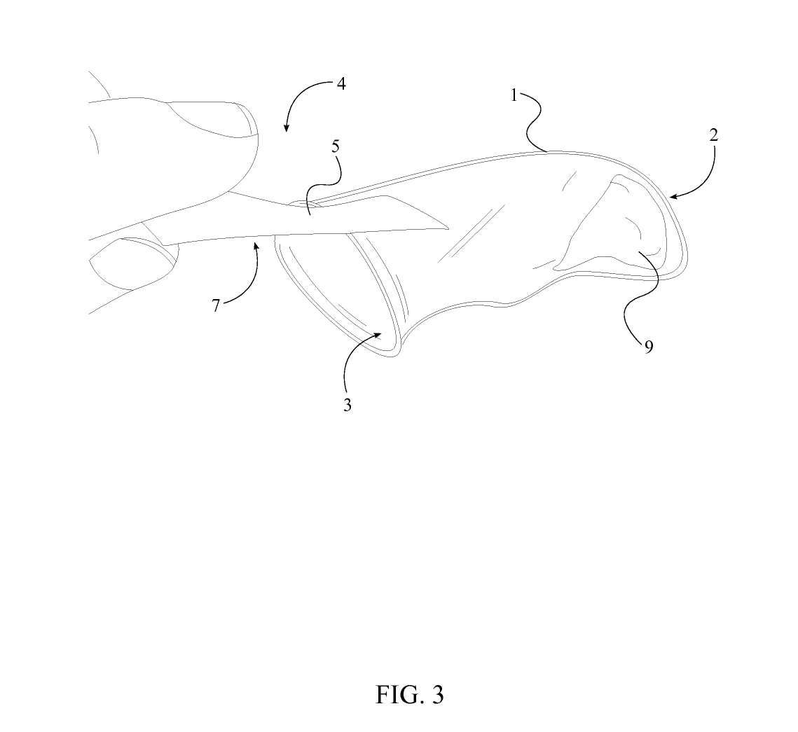

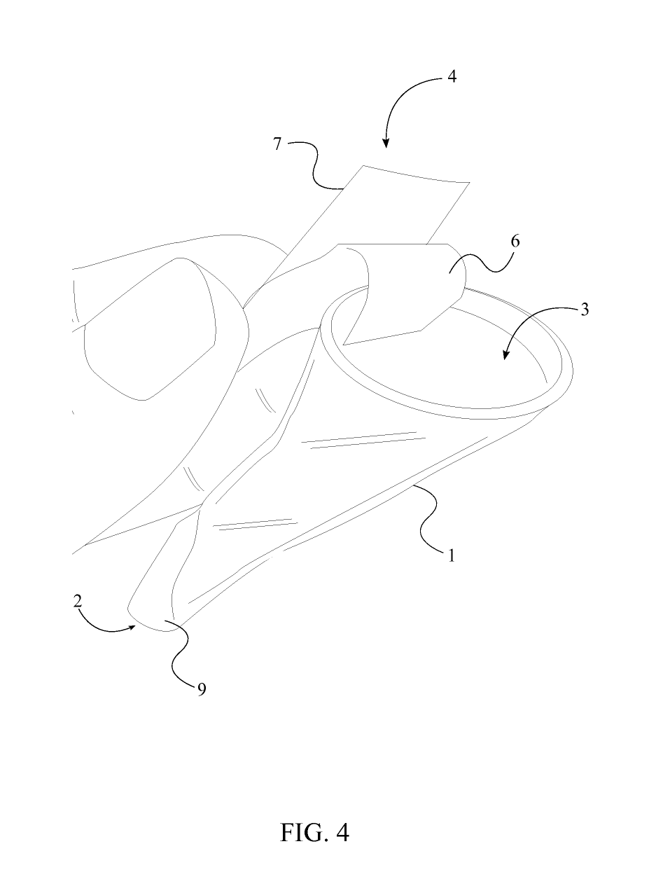

As mentioned earlier, the present invention is fastened onto the finger of the user with the attachment mechanism 4. In order to do so, the attachment mechanism 4 is externally connected to the finger cover 1 at the open end 3 in the preferred embodiment of the present invention. As illustrated in FIG. 3 and FIG. 4, the attachment mechanism 4 comprises a tab 5, an adhesive layer 6, and a peel-off layer 7. The tab 5 is adjacently connected to the finger cover 1 adjacent to the open end 3 such that the user can conveniently utilize the attachment mechanism 4 by grasping the tab 5. The adhesive layer 6 is adjacently connected across the tab 5. The finger cover 1 is secured on the finger of the user with the adhesive layer 6. Even though the present invention is secured using an adhesive layer 6, in the preferred embodiment of the present invention, the present invention can also be secured through methods that can include, but is not limited to, hook and loop fasteners. The peel-off layer 7 is utilized to protect the adhering properties of the adhesive layer 6 until the time of use. In order to do so, the peel-off layer 7 is adjacently connected across the adhesive layer 6 opposite to the tab 5.

The present invention can be worn at any time moisturizing is required, regardless of the time and place. However, some users may not desire the present invention to be visible to others when worn in public. In order to address the issue, the finger cover 1 is made of a transparent material. The transparent material reduces the possibility of the finger cover 5 being seen when compared to another embodiment wherein the finger cover 5 is made of an opaque material.

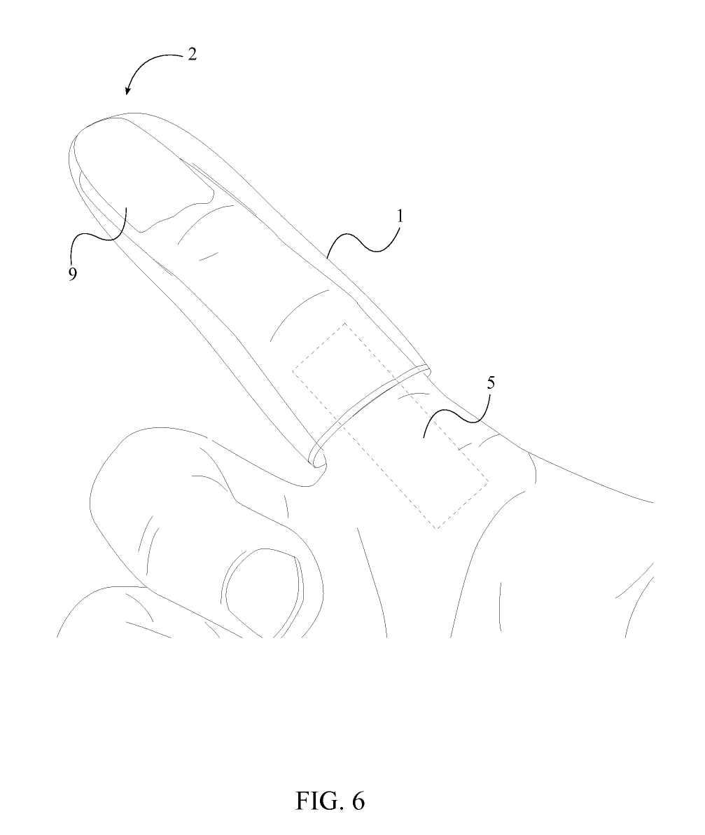

As mentioned before, the quantity of lotion 9 is positioned at the closed end 2 in the preferred embodiment of the present invention. Therefore, only the top of the finger will be in direct contact with the quantity of lotion as illustrated in FIG. 6. However, some users may prefer to spread the quantity of lotion 9 along the finger. In such instances, the user can use the transparent properties of the finger cover 1 to spread the quantity of lotion 9 along the finger. More specifically, the user can spread the quantity of lotion 9 along the finger by looking through the finger cover 1.

In order to provide user convenience, the present invention needs to be lightweight and flexible. In order to fulfill the requirements the finger cover 1 is made of a lightweight plastic. Additionally, the quantity of lotion 9 is also protected from different weather conditions since the finger cover 1 is made of the lightweight plastic. As an example, the waterproof qualities of the lightweight plastic protect the quantity of lotion 9 against rain and other environmental conditions. However, a similar material can also be used for the finger cover 1 in another embodiment of the present invention.

In utilizing the present invention, the following process flow is followed. First, the open end 3 is cleared such that the finger can be inserted through the open end 3. Afterwards, the user inserts a finger into the finger cover 1 through the open end 3. As a result, the finger comes in direct contact with the quantity of lotion 9 positioned at the closed end 2. The transparent material of the finger cover 1 allows the user to spread the quantity of lotion 9 by looking through the finger cover 1. Next, the user holds the present invention by the tab 5 and removes the peel-off layer 7. Removing the peel-off layer 7 exposes the adhesive layer 6. As illustrated in FIG. 6, the adhesive layer 6 will attach opposite to the palm of the user. By utilizing the present invention, the user can be guaranteed to have a remedy for cracked fingers.

Although the invention has been explained in relation to its preferred embodiment, it is to be understood that many other possible modifications and variations can be made without departing from the spirit and scope of the invention as hereinafter claimed.

What is claimed is:

1. An apparatus used to maintain moisture on a finger comprises:
a finger cover;
an attachment mechanism;
a quantity of lotion;
the finger cover comprises a closed end and an open end;
the closed end and the open end being positioned opposite to each other along the finger cover;
the quantity of lotion being positioned within the finger cover adjacent the closed end; and
the attachment mechanism being externally connected to the finger cover at the open end.

2. The apparatus used to maintain moisture on a finger as claimed in claim 1 comprises:
the attachment mechanism comprises a tab, an adhesive layer, and a peel-off layer;
the tab being adjacently connected to the finger cover adjacent to the open end;
the adhesive layer being adjacently connected across the tab; and
the peel-off layer being adjacently connected across the adhesive layer, opposite to the tab.

3. The apparatus used to maintain moisture on a finger as claimed in claim 1, wherein the finger cover is made of a lightweight plastic.

4. The apparatus used to maintain moisture on a finger as claimed in claim 1, wherein the finger cover is made of a transparent material.

5. The apparatus used to maintain moisture on a finger as claimed in claim 1, wherein the finger cover is a loose fitting finger cover.

6. An apparatus used to maintain moisture on a finger comprises:
a finger cover;
an attachment mechanism;
a quantity of lotion;
the finger cover comprises a closed end and an open end;
the closed end and the open end being positioned opposite to each other along the finger cover;
the quantity of lotion being positioned within the finger cover adjacent to the closed end;
the attachment mechanism being externally connected to the finger cover at the open end;
the attachment mechanism comprises a tab, an adhesive layer, and a peel-off layer;
the tab being adjacently connected to the finger cover adjacent to the open end;
the adhesive layer being adjacently connected across the tab; and
the peel-off layer being adjacently connected across the adhesive layer, opposite to the tab.

7. The apparatus used to maintain moisture on a finger as claimed in claim 6, wherein the finger cover is made of a plastic.

8. The apparatus used to maintain moisture on a finger as claimed in claim 6, wherein the finger cover is made of a transparent material.

9. The apparatus used to maintain moisture on a finger as claimed in claim 6, wherein the finger cover is a loose fitting finger cover.

10. An apparatus used to maintain moisture on a finger comprises:
a finger cover;
an attachment mechanism;
a quantity of lotion;
the finger cover comprises a closed end and an open end;
the closed end and the open end being positioned opposite to each other along the finger cover; and
the finger cover being made of lightweight plastic.

11. The apparatus used to maintain moisture on a finger as claimed in claim 10 comprises the quantity of lotion being positioned within the finger cover adjacent the closed end;
the attachment mechanism being externally connected to the finger cover at the open end;
the attachment mechanism comprises a tab, an adhesive layer, and a peel-off layer;
the tab being adjacently connected to the finger cover adjacent to the open end;
the adhesive layer being adjacently connected across the tab; and
the peel-off layer being adjacently connected across the adhesive layer, opposite to the tab.

12. The apparatus used to maintain moisture on a finger as claimed in claim 10, wherein the finger cover is made of a transparent material.

13. The apparatus used to maintain moisture on a finger as claimed in claim 10, wherein the finger cover is a loose fitting finger cover.
